(19) United States
(12) Patent Application Publication
(10) Pub. No.: US 2004/0176323 A1
(43) Pub. Date: Sep. 9, 2004

(54) Pharmaceutical composition comprising lysophophatidic acid

(76) Inventors: Dong Keun Song, Kangwon-do (KR); Sung Oh Huh, Kangwon-do (KR); Jung Sook Cho, Kyungsangbuk-do (KR); Hong Won Suh, Kangwon-do (KR); Yung-hi Hi Kim, Kangwon-do (KR)

Correspondence Address: BURNS DOANE SWECKER & MATHIS L L P, POST OFFICE BOX 1404, ALEXANDRIA, VA, 22313-1404, US

(21) Appl. No.: 10/475,814

(22) PCT Filed: Apr. 24, 2002
(86) PCT No.: PCT/KR02/00753

(30) Foreign Application Priority Data
Apr. 25, 2001 (KR) 2001/22322
Jul. 13, 2001 (KR) 2001/42395

(51) Int. Cl. A61K 31/685

(52) U.S. Cl. 514/78

(57) ABSTRACT

Provided is a pharmaceutical composition which can effectively prevent and treat sepsis and stroke. The composition comprises lysophosphatidic acid or a pharmaceutically acceptable salt thereof as an effective ingredient.

### FIELD OF THE INVENTION

The present invention relates to a pharmaceutical composition comprising lysophosphatidic acid (LPA) or a pharmaceutically acceptable salt thereof.

### BACKGROUND OF THE INVENTION

Sepsis initially accompanying extreme systemic inflammatory responses occurs when a host such as a mammal, is in the presence of an excessive systemic response to bacterial infection (for example, endotoxin of gram-negative bacteria), leading to host mortality of approximately 45%. Although antibiotics or steroids have been conventionally used for treatment of sepsis, their therapeutic effects are insignificant, presenting still high mortality of host due to sepsis.

Stroke is one of the commonest central nervous system (CNS) diseases causing abrupt coma and motor and sensory disturbances, and is one of three primary causes of human deaths together with cancers and heart disease. Stroke is classified into occlusive cerebrovascular diseases (e.g., cerebral thrombosis, cerebral embolism, etc.) and hemorrhagic cerebrovascular diseases (e.g., cerebral hemorrhage, subarachnoid hemorrhage, etc.). In particular, ischemic stroke resulting from occlusive cerebrovascular diseases takes up approximately 80% of all stroke patients.

In the case of stroke, cells in the core region that are attacked by cerebral ischemia resulting from blockage or reduction in oxygen supply due to blood circulation disturbance suffer functional disturbance within from several seconds to several minutes, causing irreversible damage. On the other hand, cells in the penumbra region attacked by cerebral ischemia undergo metabolic disturbances, but can be protected from irreversible damage if an appropriate treatment is initiated in the early stage of stroke (Choi DW (1992) Excitotoxic cell death. J. Neurobiol. 23: 1261-1276; Lipton P (1999) Ischemic cell death in brain neurons. Physiol Rev 79:1431-1568).

According to the current state of knowledge, complex mechanisms including suppressed ATP-producing capacity due to functional disorder of mitochondria, a drastic change in membrane permeability, excessive release of excitatory neurotransmitters such as glutamate, an increase of intracellular Ca²⁺, activation of Ca²⁺-dependent protease, formation of free radicals, inflammation and so on are implicated in neuronal damage due to cerebral ischemia (Lipton P (1999) Ischemic cell death in brain neurons. Physiol Rev 79: 1431-1568).

In clinical practice, various pharmaceutical agents including thrombolytic agents such as tissue plasminogen activator (TPA) or urokinase, antiplatelet agents, anticoagulants, cerebral vasodilators, Ca²⁺-channel blockages, cerebral edema inhibitors have been used for treatment of stroke (SandercoSck P, Lindley R and Wardlaw J (1992) Antiplatelet, anticoagulant and fibrinolytic agents in acute ischemic stroke and transient ischemic attack. Br. J. Hosp. Med. 47: 731-736). However, it has been known that these pharmaceutical agents have trivial therapeutic effects if therapeutic treatment is delayed and cannot effectively prevent the progress of acute cerebral ischemia into cerebral infarction (Steinberg P (1994) Stroke: The way things really are. Stroke 25: 1290-12945), while producing several adverse side effects such as nonspecific hemorrhage, fibrinogen dissolution and acute reocclusion.

Recently, many researchers have tried multilateral attempts at new causal therapeutics which can block or prevent irreversible damage based on the mechanism of neuronal damage due to cerebral ischemia. In other words, recent researches mainly aim at a development of glutamate-receptor antagonists, Ca²⁺-channel blockers, Na⁺-channel blockers, free-radical removers, calpain inhibitors, and nitrogen monoxide synthetic enzyme inhibitors (Leeson PD and Iversen LL (1994) The glycine site on the NMDA receptor: structure-activity relationships and therapeutic potential. J. Med Chem. 37: 40534067; Muir KW and Lees KR (1995) Clinical experience with excitatory amino acid antagonist drugs. Stroke 26: 503-513). As a result, several pharmaceutical agents have been developed and are currently under clinical tests. However, since the mechanism of neuronal damage is very complex and the developed pharmaceutical agents have various problems of adverse side effects or infiltration into cerebral tissues, the fact is that there is no effectively therapeutic compound developed.

The inventors of the present invention have carried out research on therapeutic use of lysophosphatidic acid (LPA) for several years and have observed that LPA has therapeutic and preventive effects for sepsis and stroke, thereby completing the present invention.

To date, it has never been reported that LPA has therapeutic and preventive effects for sepsis and stroke.

### OBJECT OF THE INVENTION

It is an object of the present invention to provide a pharmaceutical composition having good preventive and therapeutic effects for sepsis, comprising lysophosphatidic acid (LPA) or a pharmaceutically acceptable salt thereof as an effective ingredient.

It is another object of the present invention to provide a pharmaceutical composition having good preventive and therapeutic effects of stroke, comprising lysophosphatidic acid (LPA) or a pharmaceutically acceptable salt thereof as an effective ingredient.

### SUMMARY OF THE INVENTION

To accomplish the objects, an aspect of the present invention provides a composition for prevention and treatment of sepsis, comprising LPA or a pharmaceutically acceptable salt thereof as an effective ingredient.

Another aspect of the present invention provides a composition for prevention and treatment of stroke, comprising LPA or a pharmaceutically acceptable salt thereof as an effective ingredient.

### BRIEF DESCRIPTION OF THE DRAWINGS

FIG. 1 is a graph showing the average of total cerebral infarct areas in cortex and striatum of control group rats and test group rats (administered with LPA); and

FIG. 2 is a graph showing total infarct volumes in cerebral cortex and striatum of control group rats and test group rats, and the average thereof.

### DETAILED DESCRIPTION OF THE INVENTION

The invention will now be described in detail.

In the composition of the present invention, lysophosphatidic acid (LPA) used as the effective ingredient is represented by formula I:

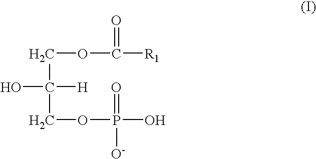

wherein R₁ is a substituted or unsubstituted straight or branched C₄₋₃₀ alkyl.

LPA can be easily commercially available. Also, LPA can be isolated from plants or animals or can be prepared by common synthesis techniques known in the art, for example, from phosphatidic acid by using phosphorlipase A.

Examples of pharmaceutically acceptable salts of lysophosphatidic acid include, but are not limited to, salts with inorganic bases such as sodium, potassium, magnesium, calcium, etc., ammonium salt, salts with organic bases such as lysine, N,N-dibenzylethylenedianine, angelic acid, etc., and so forth.

LPA and a pharmaceutically acceptable salt thereof exhibit superior preventive and therapeutic effects for sepsis, and thus significantly reduce fatality rates resulting from sepsis. Also, LPA and a pharmaceutically acceptable salt thereof remarkably suppress cerebral infarction caused by cerebral ischemia, thereby exhibiting excellent preventive and therapeutic effects of stroke.

Since LPA is an intrinsic material in a mammal, its safety is as good as proved.

The pharmaceutical composition according to the present invention can be formulated in various types for parenteral or oral administration. Examples of representative formulations for parenteral administration include isotonic aqueous solutions or suspensions as injection formulations. Examples of representative formulations for oral administration include tablets or capsules. Such formulations may further include a diluent, for example, lactose, dextrose, sucrose, mannitol, sorbitol, cellulose and/or glycine, or a lubricator, for example, silica, talc, stearic acid and a magnesium or calcium salt thereof, and/or polyethylene glycol, in addition to the effective ingredient. The tablets may further include a binder such as magnesium aluminum silicate, starch paste, gelatin, tragacanth, methylcellulose, sodium carboxymethylcellulose and/or polyvinylpyrrolidine. In some case, the tablets may include a disintegrating agent such as starch, agar, alginic acid or sodium salts thereof, or boiling mixture and/or an absorbent, a coloring agent, a flavoring agent, and a sweetening agent. The formulations are generally prepared by mixing, granulating or coating.

The pharmaceutical composition according to the present invention is sterilized and/or may further include additives such as a preservative, a stabilizer, a hydrator or emulsion accelerator, an osmosis controlling salt and/or a buffering agent, and therapeutically useful materials, and may be formulated by well known methods in the art.

As an effective component of the inventive composition, LPA and a pharmaceutically acceptable salt thereof can be administered by parenteral or oral routes once or more times daily in an amount of 0.1 to 100 mg/kg (body weight) for mammals including humans.

The present invention will now be described in more detail with reference to the following examples. However, the following examples are intended to illustrate the present invention in further detail and should by no means be construed as defining the scope of the invention.

In the following examples, all percentages in solid/solid, liquid/liquid and liquid/solid mixtures are based on percentages (%) by weight/weight, volume/volume and weight/volume, respectively, and all reactions are carried out at room temperature unless otherwise stated.

### EXAMPLE 1

Cecal Ligation and Puncture (CLP) Model Test

To verify excellent preventive and therapeutic effects of LPA as an effective ingredient of the pharmaceutical composition according to the present invention on sepsis, this test using CLP model animals in which sepsis was caused by inducing peritonitis by cecal ligation and puncture was undertaken.

After anesthetizing 15 ICR mice (weighing about 25 to 30 g; availably from MJ Ltd.) with pentobarbital, a right region of their abdomen was excised in 1 cm length to expose cecum, followed by ligating the lower area of ileocecal valve, making six punctures on the cecum with a 21 gauge needle and then suturing the abdomen, thereby inducing sepsis with the model mice.

At 2 and 4 hours after suturing, LPA(oleoyl-sn-glycerol-3-phosphate; Sigma Co.) dissolved in 10% DMSO solution was intraperitoneally administered to 5 ICR mice at a dose of about 10 mg/kg (Group A) and to another 5 ICR mice at a dose of about 50 mg/kg (Group B), and only a 10% DMSO solution was intraperitoneally administered to the other 5 ICR mice (Control group). The survival rates of ICR mice in groups A and B and control group were investigated in course of time. The results are shown in table 1 below.

TABLE 1

| | 24-hour survival rate (%) | 48-hour survival rate (%) |
|---|---|---|
| | (Mice survived/All mice) | (Mice survived/All mice) |
| Control group | 20% (1/5) | 0% (0/5) |
| Group A | 60% (3/5) | 40% (2/5) |
| Group B | 60% (3/5) | 40% (2/5) |

As shown Table 1, the mice of the groups administrated with LPA exhibited a much higher survival rate than the control group mice, confirming that LPA had preventive and therapeutic effects on sepsis.

### EXAMPLE 2

To verify excellent preventive and therapeutic effects of LPA as an effective component of the pharmaceutical composition according to the present invention on stroke, the test using rat models induced with permanent focal cerebral ischemia by occlusion of middle cerebral artery, designed as follows, was undertaken.

Rat Model Induced with Permanent Focal Cerebral Ischemia by Occulusion of Middle Cerebral Artery

12 male Sprague-Dawley rats weighing about 250 to 269 g, were anaesthetized through inhalation of a mixed solution of 70% nitrogen dioxide and 30% oxygen gas, the solution containing 2% isoflurane (Choongwae Pharma Corp., Korea), and then subjected to a slightly modified Nagasawa and Kogure's method (Nagasawa H and Kogure K (1989) Correlation between cerebral blood flow and histologic changes in a new rat model of middle cerebral artery occlusion. Stroke 20: 1037-1043), leading to occlusion of their right middle cerebral artery. Specifically, rats were anaesthetized, and then their cervix was incised along by the middle line of their neck. Then, after separately ligating their right common carotid artery and their external carotid artery carefully so as not to cause damage to their vagus nerve, the junction of their internal carotid artery were slightly cut and then a 17 mm long silicon rubber cylinder was carefully inserted thereinto, followed by ligating the internal carotid artery on the inserted cylinder. The cylinder was made of a 4-0 nylon suture (available from Nitcho Kogyo Co., Ltd., Japan) whose one end is coated about 5 mm with a mixed solution of silicon resin (available in the trade name of Xantopren; Bayer Dental) and a hardener (available in the trade name of Optosil-Xantopren Activator; Bayer Dental) in a thickness of 0.25 to 0.3 mm, and whose the other end was rounded by heat treatment. During the entire surgical operation of 15 minutes, body temperature was maintained at 37±0.5° C. using a heating pad and an incandescent electric lamp.

Effects of LPA on Rat Model with Cerebral Infarction

To determine effects of LPA on cerebral infarction, LPA(oleoyl-sn-glycerol-3-phosphate; Sigma Co.) dissolved in a 0.9% saline solution was subcutaneously administered to 4 rats of the rat models induced with permanent focal cerebral ischemia by occlusion of middle cerebral artery, at a dose of 20 mg/kg at 1 hour before the surgery, and 2 and 6 hours after the surgery, respectively (test group). To 8 rats of control group was subcutaneously administered the same amount of a saline solution at the same time period with the case of the test group rats.

Measurement of Infarcted Area and Volume

At 24 hours after surgery, the rats of the test group and the control group were decapitated, and their brains were rapidly extracted, followed by washing with a cold saline solution. Then, by cutting the brains from the position 1 mm-distant from their frontal pol, using a brain matrix (available from Harvard Apparatus Ltd., England), 7 brain sections each having a thickness of 2 mm were produced. The sections were, then, stained in 2% 2,3,5-triphenyltetrazolium chloride (TTC) in saline, for 30 minutes at 37° C., according to the procedure described by Bederson et al, (1986) Stroke 17: 1304. After the brain sections stained with TTC were fixed with 10% phosphate-buffered formalin, the area of cerebral infarcts on the posterior side of each section, which was visualized as an area of unstained tissue, was determined separately for cortex and striatum, by using an image analyzer.

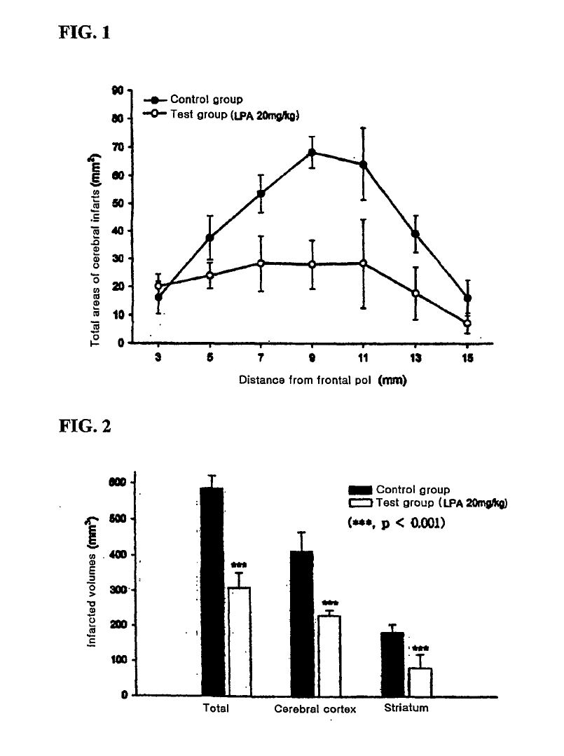

In order to correct a change in the area of cerebral infarcts due to cerebral edema, the area of cerebral infarcts on each section was determined by subtracting the unstained area in the right hemisphere with occluded middle cerebral artery from the area of the left hemisphere with unoccluded middle cerebral artery. Total areas of cerebral infarcts, obtained by summing the areas of infarcts in cortex and striatum that were measured at each brain section, were averaged, and the results were shown in FIG. 1. Referring to FIG. 1, the test group rats (administrated with LPA) exhibited a noticeable reduction in total area of infarcts compared to the control group rats.

The volume of the infracted region was calculated by multiplying the area of infarcts of each brain section with the thickness of the brain section, and determined as mean±standard deviation of data from 8 control group rats and 4 test group rats, with a significant difference evaluated by unpaired Student's t-test. FIG. 2 shows the averages of infarcted volumes in cerebral cortex and striatum of the control group rats and the test group rats, respectively and the average of total infarcted volumes thereof Referring to FIG. 2, in both cortex and striatum, the test group rats showed reductions of the infarcted volume by 44.6±3.6% and 55.3±21.0%, respectively (p<0.001), compared with the control group rats. Total infracted volume also reduced by 47.8±7.1% (p<0.001). Therefore, it is believed that LPA has the protective effect of neurons, thereby reducing infracted portions.

### Industrial Applicability

The pharmaceutical composition comprising LPA or a pharmaceutically acceptable salt thereof as an effective ingredient can effectively prevent and treat sepsis and stroke.

What is claimed is:

1. A pharmaceutical composition for prevention or treatment of sepsis, comprising lysophosphaditic acid or a pharmaceutically acceptable salt thereof as an effective ingredient.

2. (canceled)

3. A use of lysophosphaditic acid or a pharmaceutically acceptable salt thereof in preparing a pharmaceutical composition for prevention and treatment of sepsis.

4. (canceled)

5. A method for preventing and treating sepsis by administering an effective amount of lysophosphaditic acid or a pharmaceutically acceptable salt thereof.

6. (canceled)
